(19) United States
(12) Patent Application Publication
(10) Pub. No.: US 2004/0133132 A1
(43) Pub. Date: Jul. 8, 2004

(54) Interdiscal tensiometer apparatus and method of use

(76) Inventor: James L. Chappuis, Marietta, GA (US)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 10/666,502

(22) Filed: Sep. 19, 2003

Related U.S. Application Data
(60) Provisional application No. 60/411,968, filed on Sep. 19, 2002.

(51) Int. Cl. A61B 5/103

(52) U.S. Cl. 600/594

(57) ABSTRACT

An interdiscal tensiometer comprises a load measuring means for measuring load between two points and a distance measuring means for measuring distance between the two points. A method of use is also provided.

### CROSS-REFERENCE TO RELATED APPLICATION

This application is based on and claims priority to co-pending U.S. provisional patent application entitled “Interdiscal Tensiometer Apparatus and Method of Use” filed on Sep. 19, 2002 and accorded serial No. 60/411,968, which is entirely incorporated by reference.

### TECHNICAL FIELD

The invention generally relates to systems, devices, and methods related to grafting interbody segments and, more particularly, to interdiscal tensiometer apparatus and methods of use.

### DESCRIPTION OF RELATED ART

The human spine is composed of a column of thirty-three bones, called vertebra, and their adjoining structures. The twenty-four vertebrae nearest the head are separate bones capable of individual movement and are generally connected by anterior and posterior longitudinal ligaments and by discs of fibrocartilage, called intervetebral discs, positioned between opposing faces of adjacent vertebrae. The twenty-four vertebrae are commonly referenced in three sections. The cervical spine, closest to the head and often referenced as the “neck,” comprises the first seven vertebrae of the spine. The thoracic spine and the lumbar spine are below the cervical spine. Each of the vertebra include a vertebral body and a dorsal arch, which enclose an opening, called the vertebral foramen, through which the spinal cord and the spinal nerve pass. The remaining nine vertebrae below the lumbar spine are fused to form the sacrum and the coccyx and are incapable of individual movement.

Fusion of vertebral bodies may be required for any number of reasons. Most often, such fusion is necessitated when an intervertebral disk is damaged, degenerates, or otherwise becomes diseased, causing great discomfort by way of impinging on the spinal cord and/or nerve roots. When more conservative treatments and minimally invasive procedures have been exhausted, it may become necessary to surgically remove the damaged disk and fuse the associated vertebral bodies in order to restore the original spatial relationships, as well as desired stability.

Once the damaged disk has been removed, a bone graft or fusion cage packed with grafting material, or autograft bone, is placed in the intervertebral space in order to fuse the vertebral bodies together. The grafting material typically comprises bone fragments taken from the iliac crest of the patient. For the individual fragments to become one mass that will eventually fuse the vertebral bodies, the mass of fragments needs to be placed in an environment that will exert adequate force on the fragments. Research has shown that a pre-load of approximately 400N to 900N is desirable to achieve a desirable fusion outcome. As such, the size of the fusion device chosen is important to achieving fusion.

Currently, surgeons venture an educated guess when determining the size fusion device to use during such procedures. However, where the pre-load is less than the preferred range, such as when the fusion device is too small, non-union of the fusion device can result. Where the pre-load is excessive, such as when the fusion device is too large or as can occur in a severely degenerative spine, subsidence can result. Both results are undesirable and render the surgery unsuccessful.

Therefore there is a need for improved devices, systems, and/or methods that address these and/or other shortcomings of the prior art.

### SUMMARY

Interdiscal tensiometer apparatus and methods of use are provided. An embodiment of an interdiscal tensiometer briefly described, in architecture, comprises a load measuring means for measuring load between two points and a distance measuring means for measuring distance between the two points.

Methods of use of an interdiscal tensiometer are also provided. In this regard, one embodiment of such a method, among others, can be broadly summarized by the following steps: providing a pair of primary members being hingedly fixed together, each one of the pair of primary members having a contact tine; measuring a load on the contact tines; and measuring a distance between the contact tines. The contact tines are adapted to engage a pair of intervertebral bodies such that the load measuring means can measure a load therein and the distance measuring means can measure a distance therebetween.

Other systems, methods, features and/or advantages will be or may become apparent to one with skill in the art upon examination of the following drawings and detailed description. It is intended that all such additional systems, methods, features and/or advantages be included within this description, and be protected by the accompanying claims.

### BRIEF DESCRIPTION OF THE DRAWINGS

Many aspects can be better understood with reference to the following drawings. The components in the drawings are not necessarily to scale. Moreover, in the drawings, like reference numerals designate corresponding parts throughout the several views.

FIG. 1 is a side view of an embodiment of an interdiscal tensiometer.

FIG. 2 is a side view of the interdiscal tensiometer illustrated in FIG. 1 in operation.

### DETAILED DESCRIPTION OF THE PREFERRED EMBODIMENTS

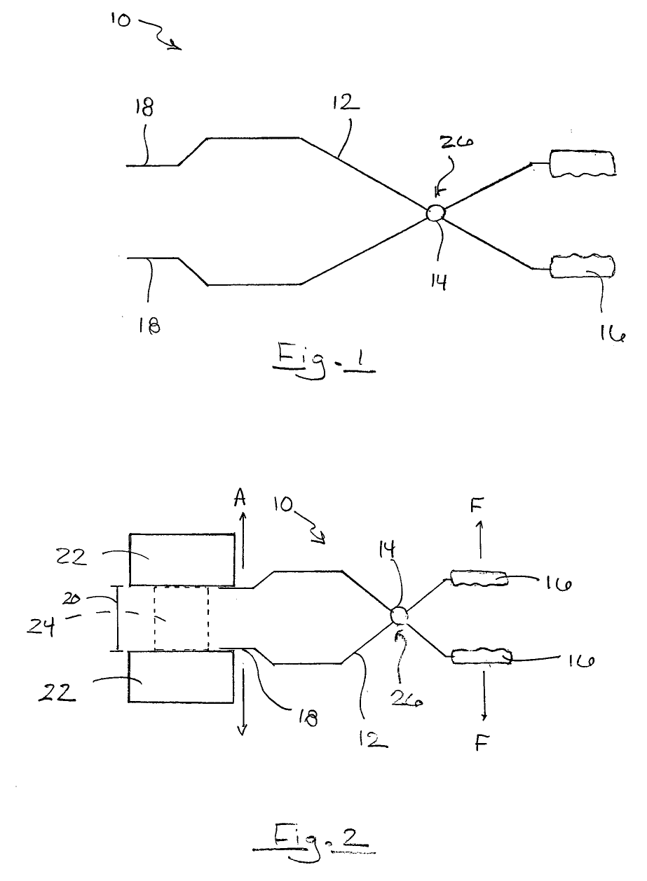

FIG. 1 illustrates one preferred embodiment of an interdiscal tensiometer (hereinafter, “tensiometer”) 10 of the present invention. The tensiometer 10 comprises a pair of substantially similar primary members 12. Each primary member 12 comprises a handle 16 and an opposing contact tine 18. The primary members 12 are pivotally fixed to each other in a cross-over configuration at a hinge connection 14. The hinge connection 14 can optionally be spring-loaded. A spring-loaded hinge connection 14 urges contact tines 18 toward each other when no force is applied to the handles 16. The spring (not shown) can be spiral, linear or any suitable configuration. As force sufficient to overcome the spring pre-load is applied to the handles 16, the contact tines 18 are urged away from each other. It should be understood that the illustrated shape of the primary members 12 is merely an exemplar shape, however, it is preferable that the primary members 12 are shaped such as to require minimal space in which to operate. It should also be understood that various shapes other than the shape depicted may be used. The primary members 12 of the tensiometer 10 can comprise any suitable material, such as, for example, stainless steel.

The primary members 12 each comprise a handle 16 disposed toward one end. It is preferable that the handles 16 provide for ease in gripping and use of the tensiometer 10. The handle 16 can comprise any suitable material, such as hard or soft rubber, plastic, or the like.

Each primary member 12 further comprises a contact tine 18 disposed opposing the handle 16. The contact tine 18 is arranged and configured to contact a portion of a vertebral body.

The tensiometer 10 further comprises a tension measure device 26. The tension measure device 26 can comprise a strain gage, or any suitable instrument for measuring load. The tension measure device can be located in any suitable location and can comprise any suitable configuration. The tension measure device 26 measures the pre-load in the interdiscal space into which the contact tines 18 are inserted. The tension measure device 26 also measures the distance disposed between the contact tines 18 when the handles 16 are urged apart. In such a configuration, the tension measure device 26 may alternately measure the distance disposed between the handles 16 when urged apart. The distance between the handles 16 then correlates to the distance measured by the contact tines 18. The distance between the contact tines 18 can be measured by any suitable measuring device that can be located in any suitable position on the tensiometer 10.

Turning next to FIG. 2, a method of use of the tensiometer 10 is illustrated. The tensiometer 10 is used to determine the appropriate size for a fusion device 24 to be disposed in an interdiscal space 20 disposed between a pair of vertebral bodies 22 in order to achieve the desired force load on the fusion device 24. The fusion device 24 can comprise a bone graft, a fusion cage packed with grafting material, autograft bone, or any suitable material and device configuration.

The contact tines 18 of the primary members 12 are disposed within the interdiscal space 20. A user grips the tensiometer 10 at the handles 16 disposed on each of the primary members 12. Portions of the primary members 12 are urged apart by application of an outward force F applied to the handles 16 of the primary members 12. The application of force F to the handles 16 pivots the primary members 12 about the hinge connection 14 causing the contact tines 18 to move apart from each other in direction A. The primary members 12 pivot about the hinge connection 14 until the contact tines 18 each engage a portion of the opposing vertebral bodies 22. Outward force F is applied to the primary members 12 until the desired force is read on the tension measure device 26. The tension measure device 26 further indicates the height measured by the contact tines 18 and indicates the size fusion device 24 appropriate for that interdiscal space.

It should be emphasized that he above-described embodiments of the present invention, particularly, a “preferred” embodiment, are merely possible examples of implementations, merely set forth for a clear understanding of the principles of the invention. Many variations and modifications may be made to the above-described embodiment(s) of the invention without departing substantially from the spirit and principles of the invention. All such modifications and variations are intended to be included herein with the scope of this disclosure and the present invention and protected by the following claim.

Therefore, having thus described the invention, at least the following is claimed:

1. An interdiscal tensiometer, comprising:
a load measuring means for measuring load between two points; and
a distance measuring means for measuring distance between said two points.

2. An interdiscal tensiometer, comprising:
a pair of primary members being hingedly fixed together, each one of said pair of primary members having a contact tine;
a tension measuring device for measuring load said contact tines;
a distance measuring device for measuring distance between said contact tines;
wherein said contact tines are adapted to engage a pair of intervertebral bodies such that said load measuring means can measure a load therein and said distance measuring means can measure a distance therebetween.

3. A method of using an interdiscal tensiometer, comprising the steps of:
a pair of primary members being hingedly fixed together, each one of said pair of primary members having a contact time;
inserting each of said contact tine between at least two vertebral bodies;
measuring a load between said at least two vertebral bodies; and
measuring a distance between said at least two vertebral bodies.
